(19) United States
(12) Patent Application Publication
(10) Pub. No.: US 2017/0361339 A1
(43) Pub. Date: Dec. 21, 2017

(54) System for effecting an exothermic reaction in a nozzle to drive a phase change from a liquid to a gas

(71) Applicant: Brian P. Roarty, Los Altos, CA (US)

(72) Inventor: Brian P. Roarty, Los Altos, CA (US)

(21) Appl. No.: 15/731,477

(22) Filed: Jun. 16, 2017

Related U.S. Application Data
(60) Provisional application No. 62/392,989, filed on Jun. 17, 2016.

(51) Int. Cl. B05B 1/00; C01D 15/06; C01B 15/14; C07C 211/63; C01B 5/02

(52) U.S. Cl. CPC B05B 1/005; C07C 211/63; C01B 5/02; C01B 15/14; C01D 15/06

(57) ABSTRACT

A device is described herein for priming and stimulating a fluid that enters a nozzle such that when the fluid experiences a phase change from liquid to gas, said phase change releases energy latent within molecules or atoms of any of the interior surface of the nozzle and the fluid, producing an energy release, thereby enabling that phase-changed gas to be used to generate energy using well-known techniques in the prior art.

### CROSS-REFERENCES TO RELATED APPLICATIONS

This application claims priority under 35 USC 119(e) from U.S. Provisional Patent Application Ser. 62/392,989 by the same inventor Brian P. Roarty, titled “A System for Effecting an Exothermic Reaction In a Nozzle to Drive a Phase Change from a Liquid to a Gas”, filed Jun. 17, 2016, which is herein expressly incorporated by reference.

### GOVERNMENT RIGHTS

None

### FIELD OF THE INVENTION

The present invention relates to a device (which comprises in part a nozzle) for making use of a fluid flowing through that nozzle, such that a phase change from liquid to gas of at least a portion of the fluid within the nozzle is driven by a release of energy latent within individual molecules or atoms within the fluid or at or within the interior surface of the nozzle. This release of energy causes the remaining liquid portion of the fluid to be heated enough locally to further undergo the phase change into a gas. Prior art fully teaches multiple alternative variations on how to provide, to recondense, and to recirculate the fluid, and how to use the gas produced within the device to generate real work.

### BACKGROUND OF THE INVENTION

The inventor has been developing this technology along two tracks. The first track is the development of a protocol for a reaction that takes place in the nozzle. One granted patent and two patent applications describe that protocol. The patent is U.S. Pat. No. 7,442,287 and the patent applications are published as US 20130233718 A1 and US 2017/0062079 A1. The second track is the development of the device and its nozzle itself.

The present application resembles a US patent application publication US 2005/0199747 A1 and a subsequent CIP published as US 2010/0123022 A1 previously submitted by the inventor, which were rejected by the U.S PTO as not being enabled. The current invention has been improved by five things that will be detailed below. The set of the granted patent, the four applications, and this current application is a body of work that collectively describes the evolution and the refinement of the invention of the device also referred to as a “Low-Energy Nuclear Reaction” (‘LENR’) phase change nozzle.

Conventional nozzles are routinely divided into two classes, one for incompressible fluids and another for compressible fluids. Each of these classes of nozzles behaves somewhat differently as fluid flows through them. In both classes, the cross-sectional area of a surface perpendicular to the fluid flow vector reduces in an inlet to a narrowest value at the throat. In the case of compressible fluids, it then increases again in an exhaust section.

The velocity of an incompressible fluid increases within the inlet section as the fluid approaches the throat and achieves a local maximum velocity at or near the throat. The increase in the velocity of the incompressible fluid is inversely proportional to the cross-sectional area of the nozzle. In contrast, under selected conditions, a compressible fluid may achieve a special value of velocity (e.g., sonic speed in the fluid) at or near the throat, while the fluid velocity may be greater than that special value in the exhaust section (e.g., supersonic speed in the fluid), where the cross-sectional area of a surface perpendicular to the fluid flow vector increases.

A liquid fluid which is incompressible may become compressible if it changes phase, either as a partial mixture of liquid and gas (e.g., wet steam) or as a completely phase-changed gas (e.g., dry steam). A nozzle arrangement whereby the fluid flowing through the nozzle is incompressible in the inlet side of the throat, and experiences a phase change to thus become compressible, with a resultant expansion in volume as it passes through the throat and through the exhaust, may manifest distinct features and advantages in application for energy generation.

Though the phase-changed material (gas, or mixture of fluid and gas) can exit directly from the throat of the nozzle, in the preferred embodiment the nozzle incorporates an exhaust section to achieve a higher velocity and effectiveness.

Prior art requires either a combustive change to the fluid, or a source of heat imported from outside the nozzle, to affect such a phase change.

A nozzle arrangement whereby the fluid flowing through the nozzle is incompressible on one side of the throat, experiences a phase change and becomes compressible as it passes through the throat into the exhaust, may manifest distinct features and advantages in energy generation and usage.

Conventionally, reactions are divided into two classes: 1) chemical reactions, and 2) nuclear reactions. Chemical reactions involve the exchange or sharing of valence electrons between atoms and result in the formation or breaking of molecular bonds. Nuclear reactions involve the subatomic particles in an atom's nucleus. They were only known to occur in three ways: a) with the natural decay of radioactive elements, b) within a plasma where the elevated temperatures strip the atoms of their valence electrons and nuclei can interact with each other without the shielding provided by the Coulomb effect of the valence electrons, and c) when atoms are bombarded by high-energy particles that can penetrate the electrons surrounding a nucleus, as in a particle accelerator.

The reactions described in b) and c) above require high-energy inputs to effect, and thus could be called High-Energy Nuclear Reactions (HENRs). HENRs produce energy densities that are approximately seven orders of magnitude above those of chemical reactions.

In contrast, the LENRs used in this invention do not require high-energy inputs. They are called “low-energy” reactions precisely because (a) they are stimulated by relatively low-energy inputs; and, (b) occur at relatively low temperatures compared to HENRs. However, LENRs produce energy densities which are closer to HENRs than to chemical reactions. One of the indications of an LENR is an energy release that cannot be created by any known chemical reaction.

Fortunately, LENRs are also believed to be surface reactions. In the case of this invention they are limited to the small volumes at or near the interior surface of the throat of the nozzle as the fluid passes through it. Those volumes could include the solid-liquid interface between the body of the nozzle and the fluid and the gas-liquid interface on the interior surface of gaseous bubbles formed within the liquid.

LENRs have been and are still sometimes conflated with “cold fusion”. Cold fusion is generally understood by people in that field to follow the Fleishman-Pons model, where deuterium atoms are loaded into the crystalline matrix of a metal such as palladium or nickel. Those deuterium atoms are thus immobilized within the matrix and thus can interact with each other in ways that are not possible as a gas.

However, the reaction described in this patent does not require the loading of either hydrogen or deuterium atoms into a material at the site of the reaction, although the liquid is primed before it enters the nozzle with a protocol that to many, and in part, resembles the Fleishman-Pons protocol.

In summary, what is described herein is a device with appropriate material properties and structure that uses a method for continuous treatment of a fluid that may then enter the nozzle as an incompressible, primed liquid upstream of, and exit as a compressible gas downstream of, the nozzle's throat, wherein an initial phase change from a liquid to a gas is induced in a minute fraction of the molecules or atoms of the fluid that effects a release of energy latent within the molecules or atoms of either the interior lining of the nozzle or of the fluid through an intra-molecular or intra-atomic, but non-combustive and non-chemical transformation. This release of energy extends the phase change to more of the fluid without necessarily causing further release of latent energy; and the gas thus generated can be used to produce useful work.

### SUMMARY OF THE INVENTION

Those needs are met by the present embodiment of the invention, which provides a device comprising a nozzle [1] comprising an inlet [2], throat [3], initiating element [8], and exhaust [5] (which in a first further embodiment widens from the throat [3]), a fluid F, and in an optional embodiment a separate priming unit in which the fluid F is first primed before it passes without any impulse drop in pressure into the inlet [2] of the nozzle [1]. The nozzle [1] is constructed to promote, effect, sustain and contain repeated or continuous energy releases from the latent energy within any of the fluid F and interior surface of the nozzle [1], and the subsequent phase change of more of the fluid F from liquid to gas. A key aspect of this invention is the initiation of an impulse drop in pressure in the fluid F as it is flowing through the nozzle [1]. The nozzle [1] initiates an initial phase change in minute fractions of the fluid F flowing through it by any combination of sonic stimulation creating collapsing bubbles in the fluid F and an impulse drop in pressure as the fluid F expands at the exhaust [5].

More specifically, the fluid F is an aqueous solution with two solutes, a lithium salt and siliceous material, that enters the throat [3] as an incompressible liquid near or above its boiling point and is converted within the nozzle [1] into a compressible gas that exits through an exhaust [5], preferably as high velocity, super-heated steam. High-velocity, super-heated steam is very useful. It can, for instance, be used to: 1) turn a turbine and generate torque, 2) distill water in an evaporator or 3) generate thrust.

The phase change from liquid to gas is driven by the release of energy from a Low Energy Nuclear Reaction (LENR) that takes place in the fluid as it passes through the nozzle [1]. That LENR reaction is enabled by priming the fluid and effecting an impulse drop in pressure, which requires relatively little energy to be introduced into the system from the surrounding environment at the moment and at the site of the reaction.

The energy of the flow out of the exhaust [5] may be transformed into mechanical energy by direct thrust from the exhaust [5], by indirect thrust where at least one exhaust [5] is off the rotational axis and points tangential to the rotational axis, by directing the exhaust [5] flow through a turbine, or through a heat exchange unit, all of which are known to the prior art. These implementations are scalable over a wide range of applications by varying the number and size of nozzle [1]s incorporated.

### BRIEF DESCRIPTION OF THE DRAWINGS

FIG. 1 is a two-dimensional, planar cutaway side view of the nozzle [1], with the fluid F (not shown) flowing from the left side to the right.

FIG. 2 is another two-dimensional, planar cutaway side view of an alternative embodiment of the nozzle [1], with the fluid F again flowing from the left side to the right, wherein the nozzle [1] is more complexly shaped.

FIG. 3 is two-dimensional, planar cutaway side view of another alternative embodiment of the nozzle [1] with a first initiating element [8] and a second initiating element [17].

FIG. 4 is a two-dimensional, planar cutaway side view of a different, alternative embodiment of the nozzle [1] which further comprises each of a heating priming element [21], an electrical priming element [23], and a photonic priming element [25].

FIG. 5 is a side view of yet another alternative embodiment where the fluid F is initially primed in a separate priming unit [31] (not shown in planar cutaway) that is external to the nozzle [1] (shown in planar cutaway), where the separate priming unit [31] is connected with the latter's inlet [2] such that the primed fluid F is transferable from the separate priming unit to the inlet [2] without any impulse drop of pressure.

FIG. 6 is a ¾ side, external view (in perspective) of a separate priming unit [31], and its respective elements.

FIG. 7 is a planar cut-away side view of the separate priming unit [31] and its interior with both its respective elements and the liquid solution therein, as they would be when the priming steps are made.

FIG. 8 is a graphical, 2-D drawing of the 3-dimensional cage structure of the silicate (TMS Polyhedral Silsequioxane) molecule on the left side, combined with the chemical formula for radicals bound to the apexes of the cage on the right side.

FIG. 9 is a datalog of a device's use showing changes in temperature at the throat [3] of the nozzle [1] as events occurred.

### DETAILED DESCRIPTION OF THE INVENTION

The needs identified above are met by the present embodiment of the invention, which provides a system with a nozzle [1] and containing a fluid F (comprising a liquid solution), both further described below. The nozzle [1] is constructed to initiate, sustain and contain repeated or continuous energy releases into and the subsequent phase change from liquid to gas within and of some portion of the fluid F. The solution that is the fluid F comprises a set of a solvent and at least one solute. The solvent portion of the fluid F is composed of any of H₂O, D₂O, and a combination of both. A lithium salt (a first solute) is dissolved into the solvent and a silicate (a second solute) is present in the fluid either in suspension or solution. In the present embodiment, the lithium salt is lithium sulfate (Li₂SO₄) monohydrate and the silicate is a soluble silsesquioxane called Polyhedral Silsequioxane hydrate-Octakis (tetramethyl-ammonium) substituted. The fluid F must be formed, primed, and in the device before the device can function. A priming element primes the fluid F initially, and an initiating element within the nozzle initiates a phase change in one or more molecules of the fluid F as it flows through the nozzle.

The summary above initially resembles the aforementioned US patent application '022. The current embodiment of the invention has been improved by these five things: 1) the identification of a soluble silicate that has been shown to decrease the priming period required for the LENR reaction to occur and thus increases the likelihood of that reaction occurring in the nozzle [1]; 2) the recognition that the LENR needed for the invention can be initiated by an impulse drop in pressure when the primed fluid F is at or above its boiling point; 3) the insight that an impulse drop in pressure will naturally occur when the primed fluid F is forced through a capillary that transitions at one point from a smaller diameter to a larger diameter, such that the resulting decrease in capillary resistance at that point lowers the pressure in the fluid F; 4) the addition of ultrasonic vibration as a stimulus in the nozzle [1] that creates cavitation bubbles within the primed fluid F, which cause an impulse drop in pressure when they collapse; and 5) the discovery that the LENR reaction does not require stimulation by electrical and photonic stimulation in the nozzle [1] itself, but rather that priming the fluid F with those two stimuli before introducing it into the nozzle [1] is sufficient to enable an LENR reaction when the fluid F passes through the nozzle [1]. That fifth point is important because it significantly reduces the complexity of the nozzle [1] and its construction, since it eliminates the need for electrical and photonic stimulating bodies in the nozzle [1] itself, such as those described in the aforementioned '022 application.

Collectively, these five improvements have proven to be enabling.

One means to prime the fluid F before it enters the nozzle [1] consists of a separate priming unit in which the fluid F is brought to the proper primed state by being heated, photonically and electrically stimulated, and then conveyed to the nozzle [1] with minimal loss of pressure. “Priming” consists of both (a) a solvent and at least one solute being introduced and mixed such that the latter dissolves into the former to form the resulting solution (the ‘fluid F’), and (b) stimulating (also called conditioning) the fluid F with photonic and electrical stimuli while it is maintained at a temperature near, but below, its boiling point. In an alternative embodiment the fluid F is primed with such stimuli in the plumbing as it is conveyed to the nozzle [1]. In another alternative embodiment the fluid F is not so heated before being conveyed to the nozzle [1], though it is introduced in such quantities that it does not significantly decrease the temperature of the portion of fluid F already in the nozzle [1].

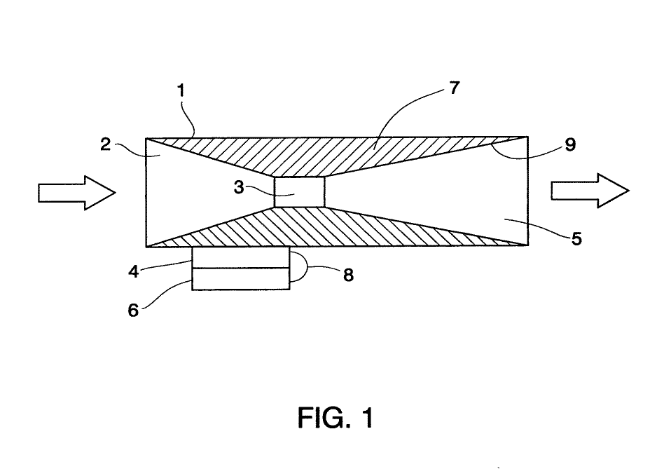

FIG. 1 shows a nozzle [1] with the fluid F (not shown) flowing from the left side to the right through an inlet [2] that narrows as it connects to a throat [3], and exhaust [5] that widens; at or near the narrow portion of the inlet [2] ultrasonic stimulation is applied to the fluid F by an initiating element [8] attached to the nozzle [1], which comprises an acoustic coupler [4] driven by a piezoelectric disc [6]. The nozzle's sidewall [7] is thick enough throughout to contain the fluid F under more than standard atmospheric pressure; and for some embodiments the nozzle's interior surface [9] that is in contact with the fluid F is any of the set of silicon, glass, ceramic or piezoelectic material, or some combination thereof.

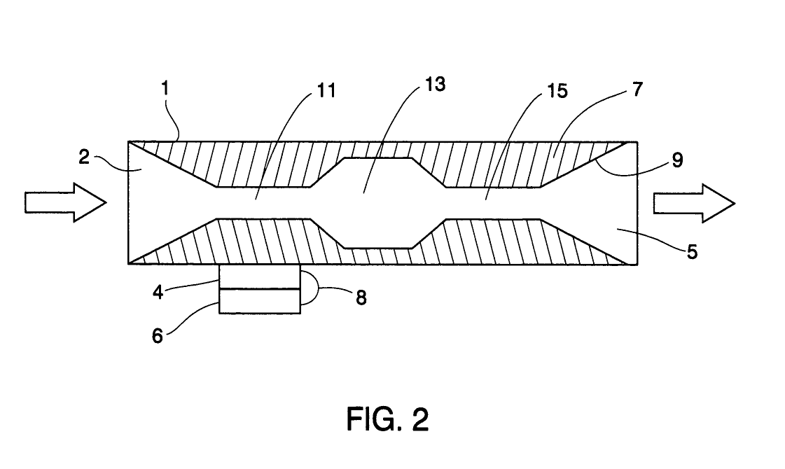

FIG. 2 shows a different embodiment where the nozzle [1] is more complexly shaped; its inlet [2] connects to a first throat [11], which connects in turn to a reaction chamber [13] which narrows again and connects in turn to a second throat [15] that connects in turn to a widening exhaust [5]; in the first throat [11] a first initiating element [8] which comprises an acoustic coupler [4] driven by a piezoelectric disc [6] applies ultrasonic stimulation to the fluid F. The interior sidewall [7] of the nozzle [1] is thick enough throughout to contain the fluid F under more than standard atmospheric pressure; and for some embodiments the nozzle's interior surface [9] that is in contact with the fluid F is any of the set of silicon, glass, ceramic or piezoelectic material, or some combination thereof.

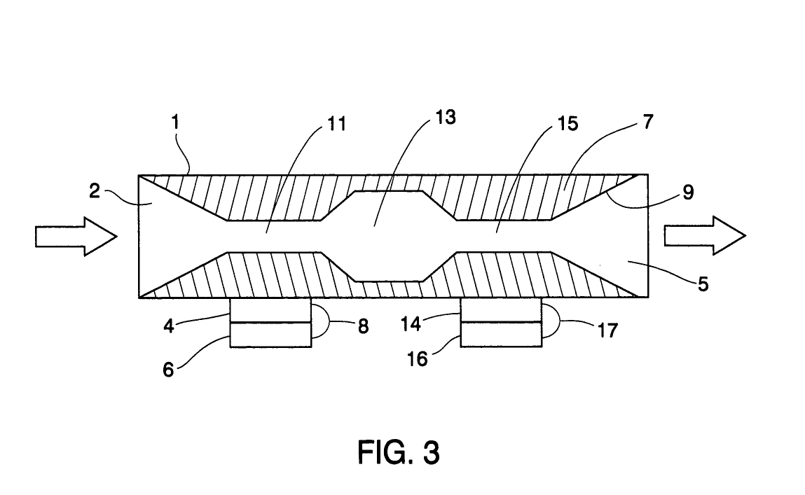

FIG. 3 shows an alternative embodiment of the nozzle [1] which, in addition to what is shown in FIG. 2, further comprises a second initiating element [17] at or near the narrowing joining of the reaction chamber [13] and the second throat [15], which second initiating element [17] is comprised of an additional acoustic coupler [14] driven by an additional piezoelectric disc [16] which applies additional ultrasonic stimulation to the fluid F.

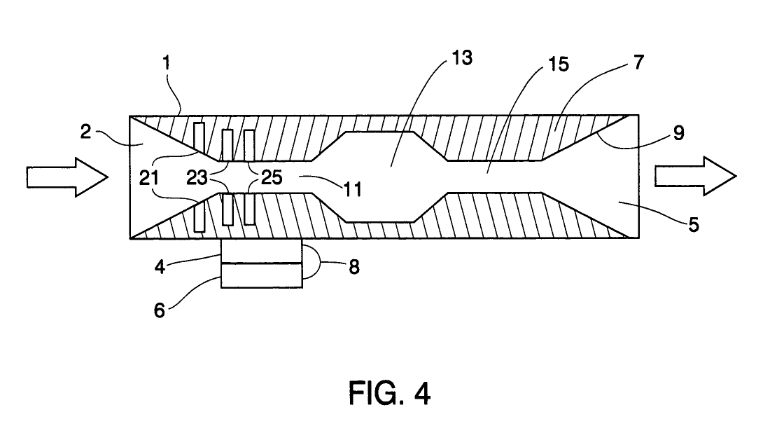

FIG. 4 shows a different alternative embodiment of the nozzle [1] from that shown in FIG. 2, in which each of a heating priming element [21], an electrical priming element [23], and a photonic priming element [25] incorporated into the nozzle [1]. The heating priming element [21] and photonic priming element [25] are spaced circumferentially around the nozzle [1] and the electrical priming element [23] is placed so it is immersed in the fluid F as that passes through the nozzle [1].

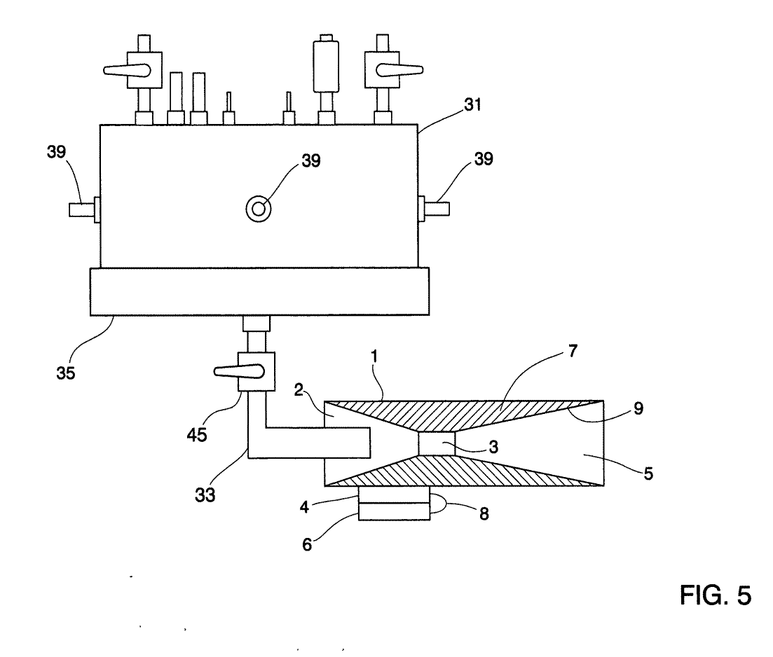

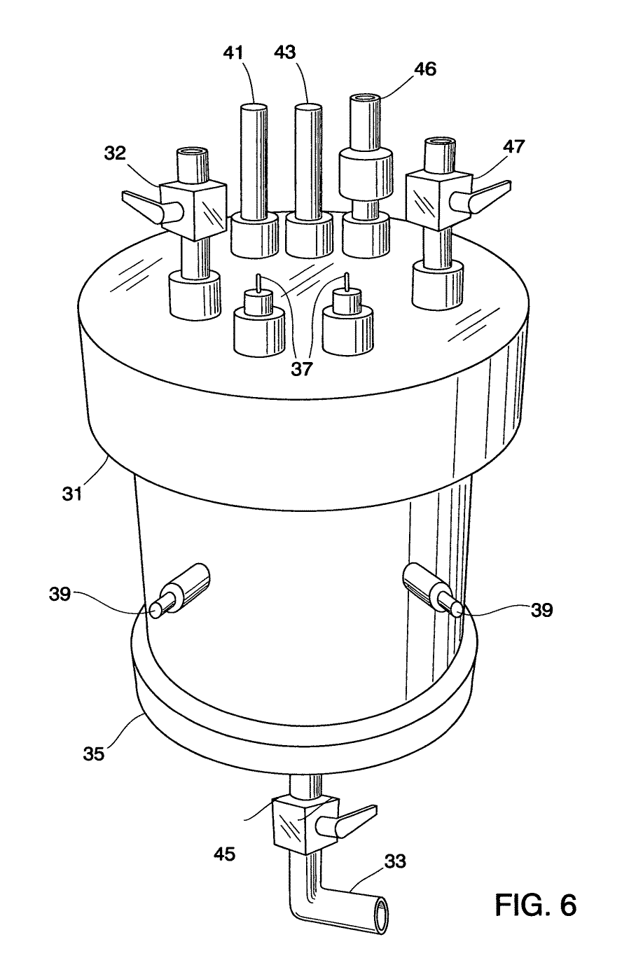

FIG. 5 shows a further alternative embodiment of the nozzle [1] than that shown in FIG. 1 where the fluid F (not shown) is initially primed in a separate priming unit [31] external to the nozzle [1], that separate priming unit [31] having a feed pipe [33] connected to the nozzle's inlet [2] such that the fluid F, having been initially primed in the separate priming unit [31] is transferred through the feed pipe [33] into the inlet [2] without any impulse drop of pressure in the fluid F. Further details of the separate priming unit are described in FIG. 6.

FIG. 6 shows the separate priming unit [31] and its constituent parts. At its top are a gas inlet valve [32], at least two electrodes [37], a first thermocouple well [41] and second thermocouple well [43], a gas relief valve [46] and a gas exhaust valve [47]. Any of the gas inlet valve [32], gas exhaust valve [47], and a liquid inlet valve (not shown as such are well known in the prior art) is used to admit any of, preferably the fluid F, and its constituent solvent and solute(s); into the separate priming unit [31]. None of the fluid F, solvent, or solutes are shown in this Figure; nor is a pressure gauge, as such is well-known in the prior art.

After the fluid F is in the separate priming unit [31], the gas inlet valve [32] can be used to admit a non-reactive gas (not shown) to fill the headspace above the level of the solution, and thus blanket the fluid F. Around the separate priming unit [31] is a separate heating priming element [35] that is used to bring the fluid F inside to, and maintain it at, a temperature within 5° C. of the fluid F's current boiling point. The first thermocouple well [41] contains a first thermocouple (not shown) that is connected with the headspace with the nonreactive gas to measure the latter's temperature; while the second thermocouple [43] well also contains a second thermocouple (not shown) that is connected with the space lower down where the fluid F is, to measure the fluid F's temperature. At least two electrodes [37] are inserted into the separate priming unit [31] with each electrode [37] extended far enough inside to be immersed in the fluid F when the device is in use.

The gas exhaust valve [46] is connected with the headspace and serves as the safety valve, reducing the pressure when that gets so high as to threaten the structural integrity of the separate priming unit [31] and any of its connecting parts.

The gas relief valve [47] is connected with the headspace and can be used to reduce the pressure or, alternatively, to allow more of the fluid F, its constituent solvent and solute(s), to replenish a decreased amount of the fluid F to be primed again.

This non-reactive gas can also move the fluid F through a feed pipe [33] controlled by a valve [45] into the inlet [2] (not shown in this Figure) with no impulse drop of pressure in the fluid F and no injection of any of the non-reactive gas into the feed pipe [33]; as it is thought better that a fractional ‘buffer amount’ of the fluid F be allowed to remain in the separate priming unit [31] when the valve [45] is closed to stop such movement of the fluid F.

Spaced (in the preferred embodiment, equally) around the side(s) of the separate priming unit [31] are a set (in the preferred embodiment, four) of separate photonic priming elements [39]. An electric stimulating means [49] (not shown in this Figure) is attached to each electrode [37].

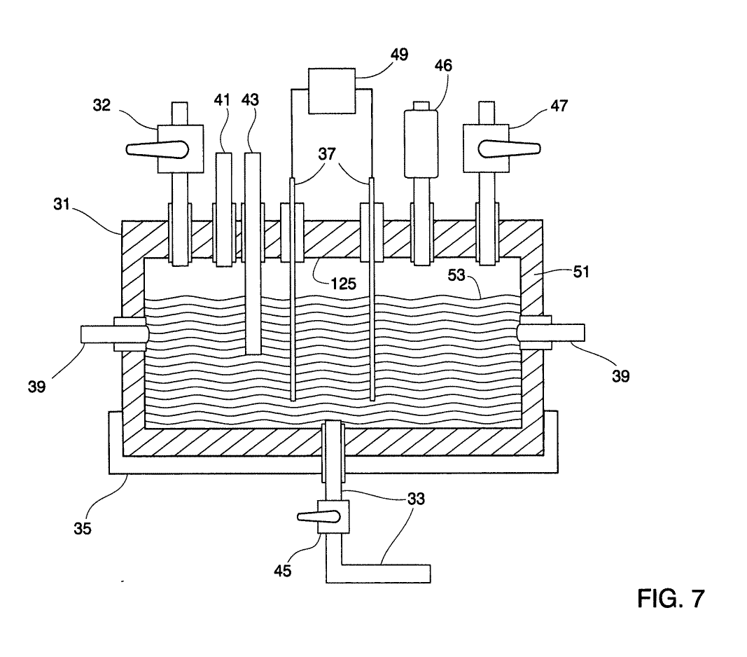

FIG. 7 shows the interior of the separate priming unit [31]. The separate heating priming element [35] is around the lower end. At the top are the gas inlet valve [32] connecting to the headspace of the interior portion; the first thermocouple well [41] also connecting to the headspace; and the second thermocouple well [43] connecting to the fluid F when the separate priming unit [31] is in use. The separate priming unit's sidewall [51] is of metal and thick enough to contain the fluid F [53] under more than standard atmospheric pressure; the sidewall's interior surface [125] which is in contact with the fluid F (or before it has become the solution, its predecessor solvent and solute(s)) is any of a glass, siliceous, and ceramic material. Also at the top the least two electrodes [37] are connecting conductively the electric stimulating means [49] and the fluid F [53] into which the at least two electrodes [37] are immersed when fluid F is being primed. Spaced around the sidewall [51] are the separate photonic stimulating elements [39]. At the bottom of the separate priming unit [31] are the feed pipe [33] that connects the interior to the inlet [2] for the nozzle [1]; and there is a valve [45] that when open, allows (but when closed, prevents) the primed fluid F to flow from the interior of the separate priming unit [31] into the nozzle [1]. The functions of the first thermocouple well [41], second thermocouple well [43], gas relief valve [46], and the gas exhaust valve [47], all remain the same as described for FIG. 6. Again, a pressure gauge is not shown.

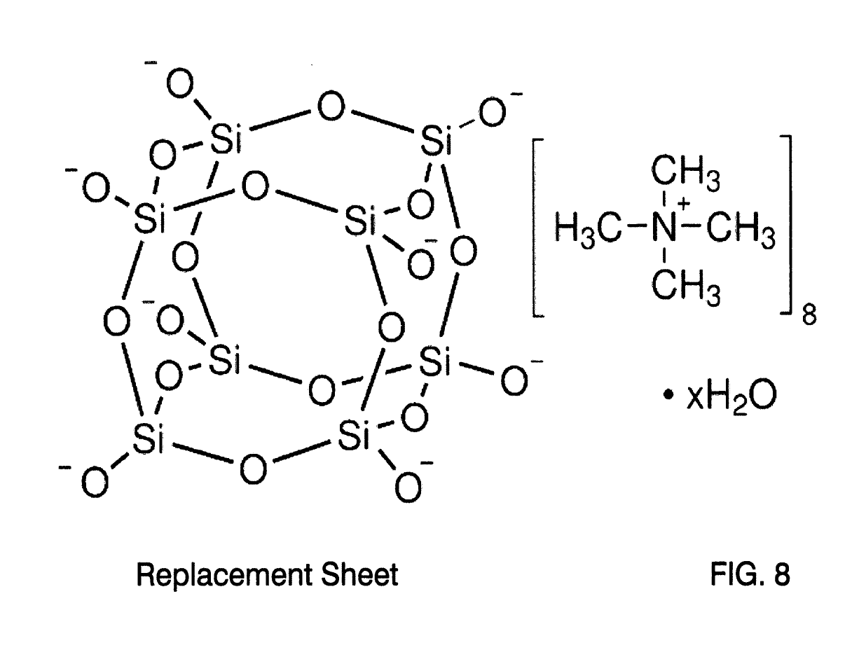

FIG. 8 is a drawing of the polyhedral silsesquioxane molecule, which is the soluble silsesquioxane (‘silicate’) described in the current application; combined with the chemical formula for radicals bound to the apexes of the molecule's central cage on the right side. The shape of the central ‘cage’ this molecule takes (again, within the inner ring of the oxygen (‘O’) and silicon (‘Si’) atoms), is more fully a three-dimensional form of two layers of rings of alternating silicon-and-oxygen atoms.

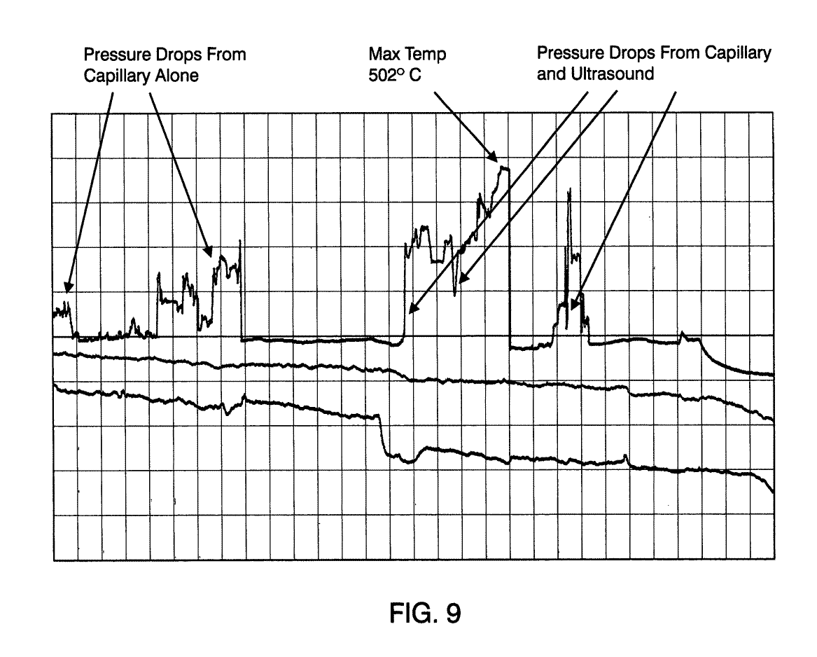

FIG. 9 shows the datalog of use of the device whereby the temperature on the outer wall at the throat of a quartz nozzle was measured by a thermocouple, shows increases of temperature in the fluid F as it passed through the nozzle. The traces for the temperature of the liquid and the headspace have a baseline of 100° C. and are scaled at 2° C. per vertical division. The baseline for the temperature at the nozzle throat is 120° C. and it is scaled at 100° C. per vertical division. The top line is the temperature at the nozzle's exhaust [5]; the middle line is the temperature of the fluid F before it enters the nozzle; and the bottom line is the temperature of the gas in the headspace of the separate priming unit above the fluid F; all temperatures being read together at the same instance. The time intervals (the x axis) are a minute per division. The profound changes (‘peaks’) that are shown correlate to the impulse drops in pressure, spiking highest when such impulse drops in pressure occur from a combination of ultrasonic initiation and capillary pressure drops together, with a maximum temperature logged of 502° C. This level of temperature change cannot be explained by any combination of chemical reaction and input energies known to the inventor.

In the present embodiment, the nozzle [1] is made of a single material: a clear fused quartz tube drawn so that it has a throat [3] having a diameter of 0.23 mm, which is narrow enough to cause capillary resistance to the flow of fluid F. Drawing the glass in this way creates a structure having the required narrowing inlet [2], a minimal-width throat [3], and an optional widening exhaust [5].

The liquid solution that is the fluid F has been found through static experiments described in the aforementioned patent application '079; in which a solution of H₂O, D₂O, Li₂SO₄, and Polyhedral Silsequioxane hydrate-Octakis (tetramethyl-ammonium) substituted (identified below as ‘Octa TMA POSS’) was able to generate LENR reactions with notably shorter gestation periods than other silicates tested by the inventor. Significantly, when a similar, superheated solution was being stimulated in a closed reactor, it was found that the desired exothermic reaction could be initiated within seconds when the pressure caused by the superheating was decreased, as detailed in the same aforementioned patent application.

The operating principle of the nozzle [1] described above is that the primed fluid F experiences a impulse drop in pressure as it passes through and exits the exhaust [5] of the nozzle [1], thus initiating a LENR reaction. In the current embodiment, two things cause the required impulse drop in pressure in the fluid F: 1) the capillary resistance decreases due to the greater radius downstream of the throat [3], and 2) the ultrasonic vibration from the acoustic coupler that is applied to the fluid F at or near the nozzle's throat [3] that generates cavitation bubbles which expand under pressure within the fluid F and subsequently collapse, resulting in an impulse drop in pressure locally at the site of the bubble. Thus, the decrease in capillary resistance would cause a continuous pressure drop, while the ultrasonic vibrations would cause an intermittent one, although in such number and with such frequency as to effectively be continuous.

Polyhedral Silsequioxane hydrate-Octakis (tetramethyl-ammonium) substituted is the molecule demonstrated to facilitate superior LENR reactions in the aforementioned '079 US application. It is manufactured by Hybrid Plastics in Hattiesburg, Miss., and has the product name “Octa TMA POSS®”, the product number MS0860, and the CAS number 69667-29-4. Hybrid Plastics defines their product as being “a hybrid molecule with an inorganic silsesquioxane at the core and anionic oxygen and a tetramethyl ammonium ion at the corners of the cage”. This chemical is also distributed by Sigma Aldrich with the part number 522260 and is labeled as having 98% purity. Interestingly, Sigma Aldrich calls this class of chemicals “Polyhedral Silsesquioxanes”, while Hybrid Plastics identifies them as “Polyhedral Oligomeric Silsesquioxanes” and has trademarked the acronym POSS®. In this application, the terms are used synonymously.

Octa TMA POSS has an interesting molecular structure. It has some attributes of both a cyclosiloxane ring and of a silsesquioxane cage structure. Specifically, it consists of two silica rings with four silicon and four oxygen atoms each. The rings are coplanar, and the radicals bound to them are tetramethylammonium, which is hydrophilic, thereby making the molecule soluble. It is known that lithium ions can bond as guests within a cyclosiloxane ring or silsesquioxane cage host, entering and leaving the center of the ring in a dynamic process that reaches a stochastic equilibrium over time. The aforementioned U.S. patent applications '718 and '079 describe protocols that exploit this “host” and “guest” phenomenon. The granted patent '287 probably also exploited that phenomenon, although the inventor did not recognize that at the time.

The Octa TMA POSS molecule used in the present invention is shown in FIG. 8. One can visualize that a lithium ion could enter the PSS molecule as a guest, much as it could a cyclosiloxane ring. However, due to the coplanar geometry, the ion would be bonded to the two rings rather than to one and, once in the center, would be held between them by the tension of being bonded in opposing directions by each of them. It would appear, then, that this host with coplanar rings would be a more stable host than a single ring, thus extending the lithium ion's status as a guest and increasing the percentage of PSS molecules hosting lithium ion guests at any given time. That may account for the lower gestation period in the static experiments.

The fluid F is first primed. This has been done in a priming unit consisting of a stainless-steel cylinder with a central well 5.08 cm deep and 5.08 cm in diameter, having a closed bottom and a removable top. A very similar reactor (as it was called there) was described previously in the aforementioned patent applications, although in this instance it was reconfigured to accommodate the feed line that conveyed the fluid from the priming unit to the nozzle [1].

As before, the described priming unit was dimensioned to accommodate a glass beaker. Alternatively, the priming unit may be a glass- or silica-lined metallic priming unit. The priming unit could also be lined with a piezoelectric material, in the form, e.g., of a porcelain glaze. The priming unit was sealed to prevent the escape of steam, along with other constituents in the solution or reaction products, or allowed only very slight escape of steam. It also allowed higher temperatures to be obtained under pressure for a given solution.

Ports in the top allowed electrodes, thermocouples, and the feed line to pass through it, while sealed glass ports in the priming unit wall allowed for the photonic stimulation by exterior illumination. The priming unit was also equipped with a pressure gauge. As a safety practice appropriate when working with exothermic reactions in a sealed priming unit at or near the boiling point of water, the priming unit was equipped with one or two pressure relief valves set to lift at several PSI above one atmosphere.

The fluid F within the priming unit is blanketed during priming with helium gas to allow the saturation of the fluid F with that gas. The helium gas was introduced into the priming unit while the atmosphere was vented through an outlet valve. The valves were ball valves made by Swagelok® ball valves, made by Swagelok Company of Solon, Ohio, that allowed for very slight release of gas. The saturation of the fluid F with this gas is optional.

A heating coil is in a cavity in the priming unit, and its input voltage and current may be measured to monitor input power. The temperature of the priming unit was raised to approximately 95° C. while the electrical and photonic stimuli were applied. A thermocouple monitored the temperature of the fluid F via a glass thermocouple well projecting into the fluid F.

Two electrodes are immersed in the fluid F. More electrodes are optional. The electrodes can be of any shape and size and must be a conductive material such as a solid metal or alloy, containing, in the current embodiment, palladium, or may be plated with a desired conductive surface material. Any of the electrodes may also be surface coated with other materials, such as silicates, with either the underlying metal or the coating or both to be treated by the protocol. One such surface treatment is shown in the SEM images included in the aforementioned '287 patent.

Electrical and photonic stimuli are applied within the priming unit. The electrical stimuli are provided via two parallel palladium electrodes of 0.063 mm diameter, a cathode and an anode. The cathode has several glass beads threaded on it, since it had previously been determined that increasing the silica content in the priming unit facilitated the LENR reaction, as noted in the aforementioned patent applications. It is presumed that the glass beads also facilitate the priming of the fluid F. The AC stimulus consisted of the beat frequency of a 3.1 MHz signal and a 43.4 MHz modulation with 9 Vpp amplitude. A concurrent DC stimulus is optional. When used in static experiments, the DC stimulus typically had 4 Volts amplitude. The DC and RF stimuli are applied from a common anode. Thus these two electrodes provided electrical stimulation to the fluid F over a time period and applied a voltage while at least one was in contact with a source of siliceous material and both were in contact with the fluid F.

The electrical stimulation may, however, consist of any of an AC voltage, a DC voltage, or both, where the AC voltage is modulated with frequencies in the RF range, in the preferred embodiment including frequencies that coincide with absorptive spectra of components of the solution and the combination of DC and AC voltages may applied any of concurrently or sequentially, between either separate anodes and a common cathode, or between a common anode and a cathode; and the electrical stimulation may be applied any of prior to, concurrent with, or subsequent to, the photonic stimulation, and so be any of concurrent and serial impulses.

The electrodes and the thermocouples are equally spaced in five holes on a bolt circle on the top of the priming unit, such that the anode was 2.3 cm away from the cathode. Similarly, the thermocouple was 2.3 cm away from the cathode. Both the electrodes and the thermocouple well passed through the priming unit's top via Teflon® PTFE seals compressed with Swagelok® fittings.

When only the AC stimulus is used, the word “cathode” is used to describe the grounded side of the AC signal, although it is not technically a cathode, since little or no net current would flow between the electrodes in the circumstance where the frequencies indicated above were used.

Four “Super Bright” white light-emitting diodes (LEDs) capable of generating 15,000 mcd were spaced equally around the priming unit below the surface of the fluid F as photonic stimuli. These photonically stimulated the fluid F through sealed glass ports in the priming unit wall. The LEDs were pulse-modulated between their on and off states with 50% duty cycles during the same period when the electrical stimulation is applied. The modulating frequency of the photonic stimuli was frequency-hopped through the following frequencies: 464; 1,234; 1,289; 2,008; 3,176; and 5,000 Hz, with each frequency hop lasting 5 seconds.

The fluid was primed for approximately four hours. After that time, it was heated above its boiling point, as shown by a positive pressure in the headspace. It was then conveyed from the priming unit to the nozzle's inlet [2] via a feed line. The feed line consisted of a 5 mm low-thermal-expansion borosilicate glass tube whose inlet [2] was immersed into the fluid. The headspace is pressurized by 1) raising the temperature of the fluid above the boiling point or 2) introducing a gas, e.g. helium, under pressure through one of the inlet valves, or 3) a combination of both of those. The feed line was curved to a right angle and a stopcock valve installed in-line to control the flow of fluid F to the nozzle [1] and to allow the pressure in the headspace to rise until the fluid F could be conveyed to the nozzle [1] in a controlled manner. The feed line terminated in a female standard taper joint and the nozzle [1] had a compatible male standard taper joint.

Several thermocouples were laid upon the feed line and the nozzle [1] itself. The feed line and nozzle [1] are then wrapped with a coating of polytetrafluoroethylene (PTFE) tape and then wrapped again with nichrome resistance wire. The nichrome wire is used to heat the feed line and the nozzle [1] body above the boiling point of the fluid F, thus ensuring that its temperature would be maintained or increase as it flowed through the feed line from the priming unit to enter the nozzle [1] at or above its boiling point. The feed line is separated into three segments: a first one between the top of the priming unit and the valve, a second one being the valve, and a third between the valve and the nozzle's inlet [2]. The nozzle [1] itself is a fourth segment. The PTFE tape insulates all four thermocouple segments from the nichrome wire. PTFE is more commonly known as Teflon®. The temperature of each of the first three heated segments is thus monitored with a separate thermocouple. The nozzle [1] itself may have multiple thermocouples, allowing the experimenter to profile the temperature along its length. Finally, the feed line and nozzle [1] are wrapped with more layers of PTFE tape to thermally insulate them and provide for more even heating.

Each segment of the nichrome wire is powered with a separate DC power supply. Temperatures are monitored on the data logger, and control of the supplies is manual.

Three things are known to stimulate LENR reactions: electricity, light, and vibration. In the aforementioned patent '287, vibration was introduced into the reaction in the form a specific modulation of the RF stimulus. In the current invention, it is introduced into the nozzle's body as ultrasonic acoustic energy. Specifically, the nozzle [1] had an acoustic coupler [4] consisting of a quartz stub extending at a right angle from the body and a circular quartz platen at the end of the stub attached to the nozzle. A piezoelectric disc transducer with a resonant frequency ranging from 1.0 to 2.5 MHz (in the present embodiment, 1.66 MHz) is bonded to the platen with a strong and stable fixative, e.g., J. B. Weld® epoxy, and excited with a waveform generator. The ultrasonic sound energy is thus conducted to the nozzle [1] and produces vibrations in the fluid F flowing through the nozzle, where it serves at least these three purposes:
It generates cavitation bubbles in the fluid F as it approaches the nozzle's throat [3]. The exact site of LENR reactions are not know at this time, however, one possible site is at the interface between the gas within a bubble and the surrounding fluid F. Cavitation bubbles thus provide more site opportunities for the reaction to occur.
Ultrasonic sound waves are known to accelerate chemical reactions. That contributes to their effectiveness in ultrasonic cleaning. Whatever process generates LENRs starts with a reaction in the domain of chemical reactions, and the ultrasonic stimulus may either accelerate those chemical reactions, or stimulate the LENR reactions, or both.
Additionally, ultrasonic bubbles in a fluid are known to generate high pressures and temperatures and, if certain conditions are met, their collapse to result in an impulse drop in pressure locally in a process called sonoluminescence. The presence of that impulse drop in pressure and heat in that collapse may also stimulate the LENR reactions.

Collectively these amount to the acoustic coupler serving as a means for inducing an impulse drop in pressure in the fluid F as it flows through the nozzle.

The selection of a piezoelectric disc [6] that will properly function with the nozzle [1] is a critical issue. Such discs are manufactured for various applications, and it was found through experimentation that one used for mist generating applications delivered enough acoustical energy that it could be effective. Such discs can be ordered online from Stiener and Martins, Inc., of Doral, Fla. The one used in the experiments reported in this application had the part number SMIST20F16RS112. It was chosen, in part, because it ships with the necessary signal leads already soldered onto it.

Piezoelectric discs have resonant frequencies, and it is known that bonding the piezoelectric disc [6] to the acoustic coupler [4] will dampen the amplitude of its vibration and may also shift the resonant frequency away from its nominal value. It is preferable to stimulate the nozzle [1] with such a resonant frequency to deliver maximum power to the fluid, and the resonant points can be verified after bonding by sweeping the disc with a waveform generator around its nominal resonant frequency and measuring the input signal with an oscilloscope. Such generators typically have variable level outputs, and they deliver a constant power once set to a given level. The resonance of the disc absorbs energy at its resonant frequency, thus causing a trough in the voltage of the stimulating waveform that is easily seen.

The ultrasonic stimulation provides other significant benefits. The ultrasonic vibration is generated in the piezoelectric disc [6] by a time-varying voltage. In turn, that voltage can have a varying amplitude. That provides a potential means of starting, stopping, and modulating the reaction. Together, those capabilities can constitute a throttle function. The ultrasonic energy also travels axially through the nozzle [1] and can be effective in stimulating the LENR reaction in the nozzle [1] upstream from where it is applied. Varying the amplitude can change the distance between the point of stimulation and the reaction site, allowing one even more control of the nozzle's performance.

Further, placing an optional second acoustic coupler [14] at a distance from the first one allows the nozzle [1] to be designed so that ultrasonic energy can be applied as stimuli from both sources simultaneously. With a nozzle's design employing the proper dimensions and ultrasonic frequencies, an antinode can be generated at a desired location within the nozzle [1], thus allowing for consistent and sustained generation of sonoluminescence at that location.

Finally, an alternative embodiment of the invention comprises a second throat [15] downstream of the first one, with a reaction chamber [13] between them. The first throat [11] would then serve to initiate an LENR reaction, possibly generating wet steam. That wet steam can mix in the reaction chamber [13] and undergo a second LENR reaction as it experiences another impulse drop in pressure flowing through the second throat [15]. This embodiment is shown in FIG. 2. Additional such stages could be added, if necessary to achieve complete phase change of the liquid to the gas phase, i.e., dry steam.

External condensation and recirculating elements, filtering, control and timing means, and mechanical energy transmission means are needed to complete the system. Such means are well known in the prior art and are neither shown nor claimed as part of this invention; however, its application and use in combination with these are not so disclaimed and may be additional parts to each of the embodiments herein.

It will be necessary to transport heat away from the nozzle [1] during operation with, for example, a fluid such as water. Again, external condensation and recirculating elements, filtering, control and timing means, and mechanical energy transmission means are needed to complete that transport. Such means are well known in the prior art and are neither shown nor claimed as part of this invention; however, its application and use in combination with these are not so disclaimed and may be additional parts to each of the embodiments herein.

### EXPERIMENTAL RESULTS

### Experiment Number 1

This experiment was an early attempt to operate a quartz nozzle and was essentially an effort to qualify the apparatus and to determine the parameters needed for proper operation. In this early implementation, the supply line did not have a valve. While heating the liquid in the priming unit and treating it with the protocol, a solid plug was inserted into the female end of the supply line to prevent steam from escaping. During the experiment, the inventor removed the plug, intending to replace it with a test nozzle, failing to notice that the fluid in the priming unit had risen above the boiling point and that the headspace was therefore pressurized. When the plug was removed, the treated liquid immediately began venting through the supply line. When it vented, the thermocouple at the end of the supply line recorded a temperature increase from 198° C. to 450° C. in less than sixty seconds. Photonic stimulation was being applied at that moment; however, the phenomenon observed in this experiment was repeated later without electrical and photonic stimuli being applied to the nozzle. These experiments established that stimulation of the fluid with those two stimuli in the nozzle was not necessary; priming the fluid as previously described herein was sufficient to enable the reaction in the nozzle under proper conditions. It is unlikely that the inventor would have ever deliberately tested under these conditions, so this is an interesting example of the potential benefits to be gained by mistakes. (Successful experiments are generally more satisfying, but we sometimes learn far more from failures than from successes.)

### Experiment Number 2

This experiment was a test of a quartz nozzle built as described in this application, including the ultrasonic stimulation. The priming unit was pressurized to 7 PSI to drive the liquid through the feed line and into the nozzle. During the experiment, fluid was observed leaking from the Teflon wrapping around the nozzle. The experiment was terminated and the nozzle later examined. It was found that it had cracked into several small pieces between 8 and 9 mm upstream of the center line of the acoustic coupler. The quartz had been tested for stress with polarized light after the glassblower worked on it and none had been detected. A severe thermal gradient would appear to have caused the strain factor that cracked the quartz at that site. Curiously, no temperature increase in the nozzle was detected in this experiment; it may simply have been so brief that a thermocouple did not have time to respond to it or that the thermocouple had not been positioned where it would have detected that increase.

### Experiment Number 3

This experiment was another test of a quartz nozzle built as described in this application, including the ultrasonic stimulation. The fluid F consisted of 30 ml of H₂O, 24 ml of D₂O, 0.190 gm TMA POSS, and 2.055 gm of Li₂SO₄. The priming unit was pressurized to approximately 9 PSI to drive the liquid through the feed line and into the nozzle. Distinct bursts of heat were recorded by a thermocouple placed on the outer wall of the nozzle at the throat. A datalog of the experiment is shown as FIG. 6. The fluid was initially passed through the nozzle without ultrasonic stimulation. That resulted in a period of temperature increases seen on the left side of the datalog. Next, the ultrasonic stimulation was added. That resulted in the steep temperature rise shown in the middle of the datalog. The ultrasonic stimulus was turned off and on three times to establish that it was causing the reaction observed, and the increases did, in fact, appear to be coincident with that stimulus. The baseline for the temperatures measured at the throat is approximately 113° C. The maximum temperature recorded is 502° C., as noted on the figure. The scan interval in this experiment was 2.5 seconds.

While the author of this application does not represent that the present embodiment of the invention is a working prototype of a phase change nozzle [1], he does assert that it is a prototype of a working prototype. Support for that assertion lies in the fact it demonstrates that the enabling exothermic reaction can be generated in the throat [3] of the nozzle [1] described herein, thus validating the operating principle of the nozzle [1] as described above.

Further, the author points to the results obtained in previous static experiments reported in the aforementioned '022 patent application. Electrodes used in those experiments were examined after testing by two different methods of elemental analysis, EDS and Auger analysis. Those examinations detected strong evidence of six different possible transmutation products of the ingredients of the system. Transmutation products imply a nuclear reaction, thus supporting the conclusion that the reaction taking place within the nozzle [1] during testing are also nuclear in nature, specifically, LENRs.

Preparation, Preferences, and Precautions (“Do's and Don'ts”)

It should be noted that this invention does not find it necessary to use D₂O (that is heavy’ as opposed to ‘ordinary’ water) to obtain an exothermic reaction. However, a solution with only D₂O yielded more frequent bursts of energy in the static experiments reported earlier in the aforementioned patent and patent applications, so there is a presumption that a mixture richer in D₂O would yield more energy with more predictability and regularity. In the event of temperature spiking or thermal blooms, control of the heat generation can be obtained through any combination of changing the amplitude of the various stimuli, concentration of any combination of the D₂O and Octa TMA POSS, or changing the diameter of any of the nozzle throat(s) [3, 11, 15].

Failure to use a silicate, and more particularly a hosting silicate, creates greater uncertainty and less success in evoking any LENR from the solution used in the device.

The selection of materials is a critical issue in the field of LENRs. For example, it has been found that the fluid F should be free from contamination by extraneous metallic particles and from particles or threads of the polymer polytetrafluoroethylene (PTFE), more commonly known as Teflon®. Although silicates and PTFE are known to be good electrical and thermal insulators, contamination by scrapings or particles of polytetrafluoroethylene appears to vitiate, dampen, or even negate any stimulated energy release and LENR. Since the static experiments reported in the aforementioned patents and patent applications were run in priming unit where the stimulating and measuring elements reached the fluid F via Teflon® seals, that material was trimmed to minimize its exposure within the system to minimize any negative effects.

Rubber has similarly been found to be a contaminant that appears to vitiate, dampen, or even negate any stimulated energy release and LENRs.

Some static experiments were conducted in a steel priming unit and attempts were made to implement a nozzle [1] made of steel. None of those experiments were successful, so others attempting to replicate this invention should be alert to the possibility that steel is also an inhibiting substance.

Another alternative embodiment of the invention comprises at least two swappable priming vessels (‘prep tank’) to effect continuous operation by enabling the substitution and simultaneous replacement of properly primed fluid F without interruption.

The use of external condensation and recirculating elements, filtering, control and timing means, and mechanical energy transmission means are well known in the prior art and are neither shown nor claimed as part of this invention; however, its application and use thereof in combination are not so disclaimed and may be embodiments herein. For example, the use of a submerged feed and pressurized headspace was chosen for simplicity and economy in the experiments reported above. A preferred embodiment would simply recirculate the fluid F with pumps; which technology is well-known to the prior art.

A number of means for transforming a heat-exchange that produces directed steam into useful work is also well known in the prior art. The common application of steam turbines and steam evaporators yield abundant evidence to support that statement. Again, the condensation, recirculation, filtering, control and timing elements are not shown; such are known to the prior art.

First among these is using the exhausted fluid in a steam jet to propel the entire system directly, according to Newton's First Law of Motion F=ma. This requires a constant replenishment of the fluid. A second means for transforming a heat-exchange that produces directed steam into useful work is to direct the exhaust into a turbine, whose spinning produces mechanical and/or electrical power. Such a turbine may, particularly at the human or larger machine scale, have vanes which are partially perpendicular to the z-axis; another alternative, feasible due to material property limitations at the smaller and micro scales is to have the steam directed across the surface(s) of the turbine's vane(s), preferably as far offset from the turbine's axis of rotation as is possible. A third means off-sets the exhaust from the z-axis and directs the exhaust tangentially to the z-axis, thereby transforming the heat-exchange which forms the steam jet into mechanical rotary motion.

While this invention is susceptible of embodiment in many different forms, there are shown in the drawings several specific embodiments, with the understanding that the present disclosure is to be considered as an exemplification of the principles of the invention and is not intended to limit the invention to the embodiments illustrated.

The above description of the invention is illustrative and not restrictive. Many variations of the invention may become apparent to those of skill in the art upon review of this disclosure. The scope of the invention should, therefore, be determined not with reference to the above description, but instead should be determined with reference to the appended claims along with their full scope of equivalents.

While the present invention has been described in connection with a preferred embodiment, these descriptions are not intended to limit the scope of the invention to the particular forms (whether elements of any device or architecture, or steps of any method) set forth herein. It will be further understood that the elements or methods of the invention are not necessarily limited to the discrete elements or steps, or the precise connectivity of the elements or order of the steps described, particularly where elements or steps which are part of the prior art are not referenced (and are not claimed). To the contrary, the present descriptions are intended to cover such alternatives, modifications, and equivalents as may be included within the spirit and scope of the invention as defined by the specification, drawings, and appended claims and otherwise appreciated by one of ordinary skill in the art.

Finally, those attempting to replicate this invention should know that, while the experiments reported herein are presented as successes, other experiments were not successful. There are many variables in LENR chemistry and it as yet is poorly understood and unpredictable. One should not expect success with every attempt; nor from failing to avoid inhibiting materials described in this application.

I claim:

1. A device for effecting an exothermic reaction to drive a phase change from a liquid to a gas, comprising:
a nozzle comprising an inlet that narrows as it connects to a throat;
a fluid solution comprising a set of a solvent and at least one siliceous solute that when combined form a fluid F, said fluid F flowing through the nozzle from the inlet to the exhaust;
a priming element that primes the fluid F with photonic and electrical stimulation to release energy latent within any of the set of atoms and molecules of any of the interior surface of the nozzle and the fluid F through a non-combustive exothermic reaction, when a phase change is initiated in one or more molecules of the fluid F within the nozzle; and,
an initiating element within the nozzle that initiates a phase change in one or more molecules of the fluid F as it flows through the nozzle.

2. A device as in claim 1 wherein the initiating element further comprises a means for inducing an impulse drop in pressure in the fluid F as it flows through the nozzle.

3. A device as in claim 2 wherein said means for inducing an impulse drop in pressure in the fluid F further comprises at least one acoustic coupler attached to the nozzle that produces vibrations in the fluid F flowing through the nozzle.

4. A device as in claim 2 wherein the nozzle throat further connects to a widening exhaust.

5. A device as in claim 1 wherein the nozzle comprises:
an inlet that narrows as it connects to a first throat;
said throat connects in turn to a widening reaction chamber;
said reaction chamber narrows again and connects in turn to a second throat; and,
said second throat connects in turn to a widening exhaust.

6. A device as in claim 5 wherein the nozzle further comprises at least one acoustic coupler attached to the nozzle that produces vibrations in the fluid F passing any of into and through at least the first throat of the nozzle.

7. A device as in claim 3 wherein the priming element further comprises a heating priming element.

8. A device as in claim 7 further wherein the heating priming element is in any of the set of priming element and nozzle, and raises and keeps the fluid F in liquid form any of at or near its boiling temperature at standard pressure.

9. A device as in claim 8 wherein the nozzle's interior surface that is in contact with the fluid F is any of the set of silicon, glass, ceramic, and piezoelectric materials.

10. A device as in claim 9, where the priming element provides any of the set of electrical and photonic stimulation to the fluid F in any of concurrent and serial impulses.

11. A device as in claim 10, wherein the priming element provides electrical stimulation to the fluid F from at least two electrodes over a time period and by applying a voltage while at least one is in contact with a source of siliceous material; and both are in contact with the fluid F.

12. A device as in claim 11, wherein the priming element photonically stimulates the fluid F through a set of white-light-emitting diodes, said diodes being:
capable of generating 15,000 mcd;
spaced circumferentially around the priming element and emitting into the fluid F;
pulse-modulated between their on and off states with 50% duty cycles during the same period when the electrical stimulation is applied;
and their modulation frequency-hopping with each frequency emission lasting 5 seconds, through the following frequencies: 464; 1,234; 1,289; 2,008; 3,176; and 5,000 Hz.

13. A device as in claim 4, wherein a separate priming unit that contains the priming element first primes the fluid F before it passes without any impulse pressure drop into the inlet of the nozzle.

14. A device as in claim 13 wherein the separate priming unit contains any set of an electrical priming element, a heating priming element, and a photonic priming element.

15. A device as in claim 14, wherein the solution comprises any of the set of heavy water (D₂O), light water (H₂O), and any mixture of heavy water (D₂O) and light water (H₂O); and the at least one solute comprises both an electrolyte including Group I element ions capable of being hosted in a hosting siliceous element, and that hosting siliceous element.

16. A device as in claim 15, wherein the electrolyte provides lithium ions capable of being hosted in a hosting siliceous element.

17. A device as in claim 16, wherein the hosting siliceous element is a polyhedral silsesquioxane.

18. A device as in claim 17, wherein:
the hosting siliceous element is a polyhedral silsesquioxane hydrate-octakis (tetramethylammonium) substituted;
the nozzle's interior surface that is in contact with the fluid F is any of the set of silicon, glass, and ceramic materials;
the priming element provides electrical stimulation to the fluid F from at least two electrodes over a time period and by applying a voltage while at least one is in contact with a source of siliceous material; and both are in contact with the fluid F;
the priming element photonically stimulates the fluid F through a set of white-light-emitting diodes, said diodes being:
capable of generating 15,000 mcd;
spaced circumferentially around the priming element and emitting into the fluid F;
pulse-modulated between their on and off states with 50% duty cycles during the same period when the electrical stimulation is applied;
and their modulation frequency-hopping with each frequency emission lasting 5 seconds, through the following frequencies: 464; 1,234; 1,289; 2,008; 3,176; and 5,000 Hz;
and,
the heating priming element raises and keeps the fluid F in liquid form any of at or near its boiling temperature at standard pressure in the separate priming element and inlet.

19. A device as in claim 12, wherein:
the solution comprises any of the set of heavy water (D₂O), light water (H₂O), and any mixture of heavy water (D₂O) and light water (H₂O); and,
the at least one solute comprises both an electrolyte including Group I element ions capable of being hosted in a hosting siliceous element, and a hosting siliceous element that is a polyhedral silsesquioxane hydrate-octakis (tetramethylammonium) substituted.

20. A device as in claim 1, further comprising power generation means attached to the nozzle's exhaust.
